(19) United States
(12) Patent Application Publication
(10) Pub. No.: US 2024/0050247 A1
(43) Pub. Date: Feb. 15, 2024

(54) STENTS FOR USE IN THE INTERVENTIONAL TREATMENT OF VASCULAR DISORDERS AND VASCULAR SURGERY

(71) Applicant: Fraunhofer-Gesellschaft Zur Forderung Der Angewandten Forschung E.V., Munich (DE)

(72) Inventors: Christian Redlich, Dresden (DE); Georg Pohle, Dresden (DE); Peter Quadbeck, Dresden (DE)

(73) Assignee: Fraunhofer-Gesellschaft Zur Forderung Der Angewandten Forschung E.V., Munich (DE)

(21) Appl. No.: 18/267,810

(22) PCT Filed: Dec. 14, 2021
(86) PCT No.: PCT/EP2021/085591

§ 371 Date: Jun. 16, 2023

(30) Foreign Application Priority Data
Dec. 17, 2020 (DE) 10 2020 216 158.5

(51) Int. Cl. A61F 2/91; A61L 31/02; A61L 31/14; A61L 31/08

(52) U.S. Cl. CPC A61F 2/91; A61L 31/022; A61L 31/148; A61L 31/088

(57) ABSTRACT

In the stent for use in the interventional treatment of vascular diseases and in vascular surgery, there is a tubular support structure formed with struts made of a first bioresorbable metallic material that are joined to one another at selective points. The surface of the struts is fully covered by a coating made with a second bioresorbable metallic material. The second metallic material has a lower dissolution rate in the implanted state under physiological conditions in the course of bioresorption, and has a more positive electrode potential compared to the first metallic material.

The invention relates to stents for use in the interventional treatment of vascular diseases and vascular surgery.

Implantable stents are used in cardiology for treating coronary vessel occlusions and in the interventional treatment of vascular diseases and vascular surgery, amongst others for treating peripheral stenoses, aneurysms, aortic dissections, or accident-related vascular lesions. In the case of coronary stents, for example, this allowed the mortality due to acute myocardial infarction to be significantly reduced. After implantation, the mechanical integrity of a stent must be ensured until the vessel remodeling has been completed. This duration is dependent on the application, the type and severity of the lesion, as well as the patient's condition. Subsequent to this functional service life, however, it is advantageous when the stent vanishes from the vessel again to avoid complications later on, such as restenosis or thrombosis.

This is not possible with stents made of traditional materials, such as steel. For this reason, bioresorbable magnesium-based or polymer-based (for example, poly-L-lactide) stents were developed and introduced in the market. Due to the low rigidity and strength of these materials, however, stent struts that have relatively large dimensions (≥100 μm), compared to commercially available stents made of medical stainless steel or CoCr, and strut widths of 60 to 80 μm are required. Yet, current publications show that the polymer stents available thus far offer no advantage over conventional stents, but, conversely, cause higher mortality due to higher thrombosis rates. Especially scaffolds that dissolve too quickly and excessively high strut thicknesses are considered to cause these surprising results. Additionally, the degradation products of resorbable polymers are known to lead to undesirable inflammatory reactions. Furthermore, the metal magnesium does not degrade evenly, which makes it more difficult to predict the change in stent properties over time after implantation.

A great need therefore exists for stents made of a resorbable material having optimized strength and degradation properties. This relates in particular to stents that are to be resorbed quickly following a functional service life that is adapted to the application (time period of mechanical integrity).

It is therefore the object of the invention to provide options that allow the properties of implanted bioresorbable stents to be defined, and in particular the progression of the degradation and resorption over time to be influenced, taking mechanical integrity into consideration.

According to the invention, this object is achieved by a stent having the features of claim 1. Advantageous refinements and embodiments can be implemented with features set out in the dependent claims.

Within the meaning of the invention, a material is referred to as bioresorbable when the material can be degraded in the body, and the degradation products can either be directly eliminated from the body or used during the course of regular metabolic processes or converted into forms that the body can use. An implant or a part of an implant that is made of a bioresorbable material loses the original shape thereof over time after implantation. During degradation, the concentrations of the elements contained in the material may exceed normal ranges in the body. After the material has been completely degraded at the site of the implantation, the concentrations decrease to the normal ranges again.

The solution according to the invention is a stent that is produced with two bioresorbable metallic materials.

The stent according to the invention has a tubular support structure, which is formed of struts that are connected to one another and made of a first bioresorbable metallic material. A coating, which is produced with a second metallic bioresorbable material, is created on the surface of the struts. The second metallic material has a lower dissolution rate under physiological conditions when implanted during bioresorption and a more positive electrode potential than the first metallic material. In the event that a first and a second metallic material have a negative electrode potential relative to a common reference electrode (for example, standard hydrogen or calomel electrode), the absolute value of the electrode potential of the second metallic material is therefore lower than the absolute value of the electrode potential of the first metallic material. Under physiological conditions, the electrode potential of the second metallic material should preferably be +150 mV higher than the electrode potential of the first metallic material to achieve as great a galvanic effect as possible.

The sum of the volume of the second metallic material is less than the volume of the first metallic material that is used to produce the stent struts.

The first metallic material can advantageously be tungsten, molybdenum, or a base alloy of one of these two metals. At least one metal contained in a molybdenum base alloy can be selected from W, Re, Nb, Ta and Mn, or at least one metal contained in a tungsten base alloy can be selected from Mo, Re, Nb, Ta and Mn.

A molybdenum base alloy comprises at least 50 at % Mo, and a tungsten base alloy comprises at least 50 at % tungsten. These metallic materials have very high strength and rigidity, making it possible to produce thin stent struts. The materials are moreover characterized by a consistent removal across the entire surface due to corrosion or bioresorption. Since the alloying elements W, Ta and Nb can be mixed with Mo using any ratio, one or more of these elements can be present in a molybdenum base alloy in an arbitrary content of greater than 0 at % and less than 50 at %. The same applies to the alloying elements Mo, Ta and Nb in a tungsten base alloy. Moreover, a Mo base alloy may contain more than 0 at % and a maximum of 42 at % rhenium, and more than 0 at % and a maximum of 36 at % manganese. A W base alloy may contain more than 0 at % and a maximum of 37 at % rhenium, and more than 0 at % and a maximum of 20 at % manganese.

A second metallic material can be pure rhenium. It is also possible to use an alloy of rhenium with molybdenum, serving as the second metallic material, which contains more than 0 at % and a maximum of 14 at % molybdenum, or an alloy of rhenium with tungsten which contains more than 0 at % and a maximum of 20 at % tungsten. It is also possible to use an alloy of molybdenum and rhenium, serving as the second metallic material, which contains more than 0 at % and a maximum of 42 at % rhenium, or an alloy of tungsten and rhenium, this alloy containing more than 0 at % and a maximum of 37 at % rhenium. If the first metallic material is an alloy including rhenium, the second metallic material has a larger rhenium content and a more positive electrode potential than the first metallic material. If the first metallic material is tungsten or a tungsten base alloy without rhenium, the second metallic material may also be pure molybdenum.

The second metallic material can be applied to the first metallic material by means of a variety of coating methods. Examples are methods of chemical vapor deposition (PVD), such as atomic layer deposition, or physical vapor deposition (CVD), such as magnetron sputtering or ion beam sputtering.

The first metallic material should be covered completely by the second metallic material across the entire surface area to prevent rapid corrosion of the first metallic material and ensure the mechanical integrity of the stent during the functional period.

The thickness of the coating that has been created with the second metallic material should be selected in the range of 1 nm to 1000 nm, and preferably 1 nm to 50 nm. The coating with the second metallic material is preferably carried out such that the layer thickness is irregular across the stent surface. The thickness of the coating should be created taking into consideration the dissolution rate of the metallic materials due to bioresorption and electrochemical corrosion as well as the time required for the restoration of the vessel wall. Part of the coating may already have degraded at the point in time at which the particular vessel wall has reached a sufficiently healthy state, as long as this does not jeopardize the mechanical integrity of the stent. For example, the health and the age of a patient prior to surgery, the type of vessel receiving the implanted stent, as well as the type and severity of the lesion can be considered for the required thickness of the coating with the second material.

The coating can be applied either to the electropolished surface of the first metallic material or after a separate surface structure has been imparted to the particular surface regions of the first metallic material. This primarily relates to the surface regions of the struts of the stent that are arranged or oriented in the vessel wall direction. These can be provided with a surface structuring produced with elevations and depressions.

The surface of the struts may have been increased with the surface structuring by a factor of 1.1 to 10 compared to an electropolished surface of the struts.

The surface structuring can advantageously have been created periodically and/or using grooves, troughs or valleys, serving as depressions, and/or elevations, using rings and/or peaks.

The surface structuring can be achieved by a locally defined material removal, preferably in the region of the surface of the struts of the stents that face the vessel wall, under the action of laser radiation or photolithography techniques. The surface structuring can also be created by etching a stent structure in a defined manner on all sides or by a tube made of a first metallic material, such as hydrogen peroxide, used as a semi-finished product.

Due to the stent being made up of two bioresorbable metallic materials, it is possible to set a resorption behavior under physiological conditions that is favorable for interventional cardiology or vascular surgery, and to set the time until the metallic materials are completely dissolved.

The dissolution of the stent according to the invention implanted in the vessel is characterized by three temporal segments having differently high dissolution rates. The duration of the time segments can, in particular, be regulated by way of the thickness of the coating, so that the dissolution behavior of the stent can be easily adapted to the particular application.

The dissolution rate is low during the first time segment since only the slowly degradable second metallic material is exposed and being resorbed. The mechanical properties of the stent are therefore constant during this time segment, which is to correspond to the functional service life, since the properties are primarily determined by the first metallic material, which is being protected against degradation during this time period.

The second time segment begins when the first metallic material has been partially exposed as a result of the degradation of the second metallic material. The partial exposure can be promoted by an irregular thickness of the coating on the surface of the struts with the second metallic material. When both metallic materials are simultaneously exposed, the differing electrode potential during this time segment cause galvanic corrosion, and the degradation of the first metallic material is locally accelerated in the surface regions in which the second metallic material has been removed, while the second metallic material is locally protected against further corrosion. Additionally, the degradation of the first metallic material is accelerated by the surface roughness/structuring, taking into consideration the degradation of molybdenum and tungsten which takes place particularly evenly across the entire exposed surface area.

The dissolution rate during the third time segment is lower than during the second time segment since the influence of galvanic corrosion becomes weaker as a result of the advanced dissolution or the fragmentation of the coating made of the second metallic material. The surface roughness/structuring, however, continues to positively affect the dissolution rate due to the larger surface. The dissolution rate is significantly higher than during the first time segment since the dissolution rate of the first metallic material is generally higher than that of the second metallic material.

The invention will be described in more detail hereafter by way of example.

In the drawing:

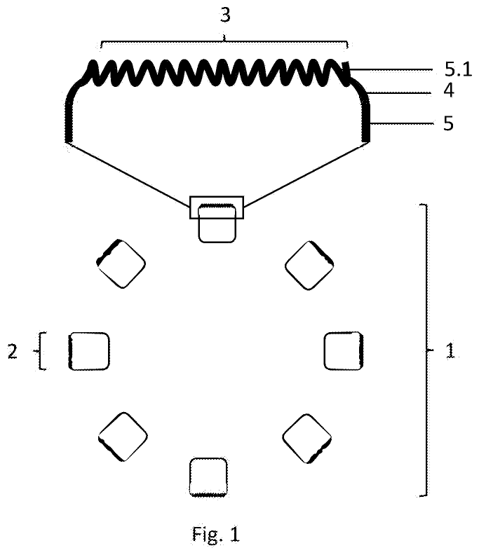

FIG. 1 shows a cut top view onto one example of a stent according to the invention in an enlarged partial illustration.

FIG. 1 shows a cut top view in a plane that is oriented perpendicular to the center longitudinal axis of the stent 1. The stent 1 is produced with struts 2, which are connected to one another at selective points (not shown). Clearances are present between the struts 2, as is also the case with conventional stents 1.

As can in particular be derived from the enlarged partial illustration shown above, the surface region of the struts 2 which faces the vessel wall has been provided with a surface structuring 3, which has been produced with grooves, serving as depressions, and rings, serving as elevations. The grooves and rings have been designed to be periodically recurring in this example.

A coating 5.1, which is produced with the second metallic material 5, is created on the struts 2, which are made of the first metallic material 4.

The dimensioning of all elements of the stent 1 can be selected in accordance with the information provided in the general part of the description.

### Exemplary Embodiment 1

This exemplary embodiment describes a cardiovascular stent 1. FIG. 1 shows a schematic sectional illustration of one example of a stent 1 comprising multiple struts 2, which are connected to one another at selective points in a manner that is not shown, wherein the connecting points are arranged in planes that are spaced apart from the plane of the shown section. The first metallic material 4 of which the struts 2 are made is pure molybdenum. This structure is generated by obtaining a small molybdenum tube having a diameter of 3 mm and a wall thickness of 50 μm using common drawing methods. The wall thickness results in the strut thickness (radial extension) of the stent structure later on. This small tube is subsequently used to produce a stent structure having a length of 30 mm by means of a laser cutting method, wherein the strut width (tangential extension) of the struts 2 connected to one another at selective points is 50 μm.

Thereafter, the structure produced with the struts 2 is cleaned and deburred by means of an electropolishing method. Using an ultrashort pulse laser, a surface structuring 3 including grooves, serving as depressions, along the length of the struts 2, having an average structure height of 5 μm, is generated on the surface of the struts 2 which faces the vessel wall, and thereby the overall surface is increased by a factor of 2.5. Thereafter, a defect-free coating 5.1 made of pure rhenium, serving as the second metallic material 5, having an average layer thickness of 20 nm is applied by means of the atomic layer deposition method. The coating 5.1 completely encloses the core of the struts 2, which are made of the first metallic material 4. Rhenium has a dissolution rate of 50 nm per year. The thickness of the coating 5.1 is selected such that the mechanical integrity of the stent 1 is ensured for the necessary functional duration of 4 months by protecting the molybdenum against corrosion. The thickness of the coating 5.1 varies slightly, in particular in the surface-structured region 3 of the struts 2.

After the functional duration has ended, during which the vessel wall of a patient has completely healed, the rhenium is dissolved locally in multiple points of the struts 2, so that the molybdenum therebeneath is exposed. This is preferably carried out in the surface-structured region 3 since regions of the coating 5.1 having varying thicknesses have been created here. The formation of local galvanic elements accelerates the corrosion of the exposed molybdenum, while the surrounding rhenium is protected against further corrosion. Due to the locally limited action of the galvanic elements, further galvanic local elements form across the entire surface of the stent structure over time.

The increased surface in the structured region 3 ensures additional acceleration of the dissolution and resorption of the stent 1. The local corrosion additionally weakens the integrity of the stent 1. Ultimately, this causes the stent structure to break, which in turn results in an increase in the surface, and thus in enhanced dissolution of the molybdenum. Without the influence of galvanic corrosion, the dissolution rate of molybdenum is 25 μm per year. The duration for the complete degradation of this stent 1 is approximately one year.

### Exemplary Embodiment 2

The exemplary embodiment describes a peripheral stent for leg arteries. A small tube is produced from a molybdenum alloy having a 25 at % tungsten content with a diameter of 5 mm and a wall thickness of 70 μm, using common drawing methods. The wall thickness results in the strut thickness (radial extension) of the stent structure later on. This small tube is subsequently used to produce a stent structure having a length of 50 mm by means of a laser cutting method, wherein the strut width (tangential extension) of the struts 2 connected to one another at selective points is 60 μm. Thereafter, the surface of the struts 2 is cleaned and deburred by means of an electropolishing method. Thereupon, the entire surface is increased by a factor of 1.5 as a result of roughening by means of etching with hydrogen peroxide. Thereafter, a defect-free coating 5.1 made of pure rhenium 5 having an average layer thickness of 5 nm is applied by means of the atomic layer deposition method. The coating 5.1 completely encloses the core of the struts 2. Rhenium, serving as the second metallic material 5, has a dissolution rate of 50 nm per year. The thickness of the coating 5.1 is selected such that the mechanical integrity of the stent is ensured for a duration of at least one month in that the coating 5.1 protects the molybdenum tungsten alloy, which serves as the first metallic material 4, against corrosion. After the functional duration has ended, within which the vessel wall of the patient has completely healed, local galvanic elements form with the molybdenum tungsten alloy therebeneath as a result of local exposure of the rhenium due to dissolution. The corrosion of the molybdenum tungsten alloy is accelerated, while the surrounding rhenium is protected against further corrosion. Due to the locally limited action of the galvanic elements, nevertheless further local elements form across the entire surface of the struts 2 of the stent 1 over time. The local corrosion weakens the integrity of the stent 1. Ultimately, this causes the stent structure to break, which in turn results in an increase in the surface, and thus in enhanced dissolution of the molybdenum-tungsten alloy. Without the influence of galvanic corrosion, the dissolution rate of the molybdenum tungsten alloy is 35 μm per year. The duration for the complete degradation of this stent 1 is approximately one year.

### Exemplary Embodiment 3

This exemplary embodiment describes a flow diversion stent 1 for treating aneurysms. FIG. 1 shows a schematic sectional illustration of one example of a stent 1 comprising multiple struts 2, which are connected to one another at selective points in a manner that is not shown, wherein the connecting points are arranged in planes that are spaced apart from the plane of the shown section. The first metallic material 4 of which the struts 2 are made is pure tungsten. This structure is generated by obtaining a small tungsten tube having a diameter of 3 mm and a wall thickness of 50 μm, using common drawing methods. The wall thickness results in the strut thickness (radial extension) of the stent structure later on. This small tube is subsequently used to produce a stent structure having a length of 30 mm by means of a laser cutting method, wherein the strut width (tangential extension) of the struts 2 connected to one another at selective points is 50 μm.

Thereafter, the structure produced with the struts 2 is cleaned and deburred by means of an electropolishing method. Using a photolithography method in conjunction with hydrogen peroxide etching, a surface structuring 3 including defined periodic depressions along the length of the struts 2, having an average structure height of 2 μm, is generated on the surface of the struts 2 which faces the vessel wall, and thereby the overall surface is increased by a factor of 1.5. Thereafter, a coating 5.1 made of a second metallic material 5, which is a rhenium alloy having a 15 at tungsten content, having an average layer thickness of 600 nm is applied by means of the magnetron sputtering method using a rotating substrate. The coating 5.1 completely encloses the core of the struts 2, which are made of the first metallic material 4. The rhenium-tungsten alloy has a dissolution rate of 3000 nm per year. The thickness of the coating 5.1 is selected such that the mechanical integrity of the stent 1 is ensured for the necessary functional duration of 2 months by protecting the tungsten against corrosion. The thickness of the coating 5.1 varies, in particular as a result of being produced by means of magnetron sputtering, in the surface-structured region 3 of the struts 2.

After the functional duration has ended, during which the vessel wall has healed and the aneurysm of a patient has been completely occluded, the rhenium tungsten alloy is dissolved locally in multiple points of the struts 2, so that the tungsten therebeneath is exposed. This is preferably carried out in the surface-structured region 3 since regions of the coating 5.1 having varying thicknesses have been created here. The formation of local galvanic elements accelerates the corrosion of the exposed tungsten, while the surrounding rhenium tungsten alloy is protected against further corrosion. Due to the locally limited action of the galvanic elements, nevertheless further galvanic local elements form across the entire surface of the stent structure over time.

The increased surface in the structured region 3 ensures additional acceleration of the dissolution and resorption of the stent 1. The local corrosion additionally weakens the integrity of the stent 1. Ultimately, this causes the stent structure to break, which in turn results in an increase in the surface, and thus in enhanced dissolution of the tungsten. Without the influence of galvanic corrosion, the dissolution rate of tungsten is 40 μm per year. The duration for the complete degradation of this stent 1 is approximately 6 months.

1-16. (canceled)

17. A stent for use in the interventional treatment of vascular diseases and vascular surgery, in which a tubular support structure, which is formed of struts that are connected to one another at selective points and made of a first bioresorbable metallic material, is formed and
a completely covering coating, which is produced with a second bioresorbable metallic material, is created on the surface of the struts,
the second metallic material having a lower dissolution rate under physiological conditions when implanted during bioresorption and a more positive electrode potential compared to the first metallic material.

18. The stent according to claim 17, wherein the surface of the struts, which are made of the first bioresorbable metallic material, is partially or completely provided with a surface structuring, which is produced with elevations and depressions.

19. The stent according to claim 18, wherein the surface of the struts, which are made of the first bioresorbable metallic material, is increased with a surface structuring by a factor of 1.1 to 10 compared to an electropolished surface of the struts.

20. The stent according to claim 17, wherein the surface structuring is created periodically and/or using grooves, troughs or valleys and/or elevations, using rings and/or peaks.

21. The stent according to claim 17, wherein the first metallic material is tungsten, molybdenum, or a base alloy of one of these two metals, at least one metal contained in a molybdenum base alloy being selected from W, Re, Nb, Ta and Mn; or at least one metal contained in the tungsten base alloy being selected from Mo, Re, Nb, Ta and Mn.

22. The stent according to claim 17, wherein the first metallic material is made of a molybdenum base alloy which contains at least 50 at % Mo or a tungsten base alloy which contains at least 50 at % tungsten.

23. The stent according to claim 22, wherein a molybdenum alloy is produced with at least one alloying element which is selected from W, Ta, Nb, Re and Mn and comprises the alloying element(s) W, Ta and/or Nb in a content of greater than 0 at % to less than 50 at % and/or the alloying element Re in a content of greater than 0 at % to 42 at % and/or the alloying element Mn in a content of greater than 0 at % to 36 at %.

24. The stent according to claim 22, wherein a tungsten alloy is produced with at least one alloying element which is selected from Mo, Ta, Nb, Re and Mn and comprises the alloying element(s) Mo, Ta and/or Nb in a content of greater than 0 at % to less than 50 at % and/or the alloying element Re in a content of greater than 0 at % to 37 at % and/or the alloying element manganese in a content of greater than 0 at % to 20 at %.

25. The stent according to claim 17, wherein the second metallic material is made of rhenium or a base alloy of rhenium, molybdenum or tungsten, at least one metal contained in the rhenium base alloy being selected from W and Mo or the metal contained in a molybdenum or tungsten base alloy being Re.

26. The stent according to claim 25, wherein a rhenium base alloy having a content of greater than 0 at % to 14 at % of Mo or a content of greater than 0 at % to 20 at % of W is produced.

27. The stent according to claim 26, wherein a molybdenum base alloy having a Re content of greater than 0 at % to 42 at % is produced, the content of Re being greater than the Re content in the first metallic material when this first material (4) is a MoRe alloy.

28. The stent according to claim 26, wherein a tungsten base alloy having a Re content of greater than 0 at % to 37 at % is produced, the content of Re being greater than the Re content in the first metallic material when this first material (4) is a WRe alloy.

29. The stent according to claim 17, wherein the second metallic material is pure molybdenum, if the first metallic material is produced with tungsten or with a tungsten base alloy which contains Mo, Ta, Nb and/or Mn.

30. The stent according to claim 17, wherein the sum of the volume of the second metallic material is less than the volume of the first metallic material (4) that is used to produce the stent struts.

31. The stent according to claim 17, wherein the coating is created with a layer thickness in the range of 1 nm to 1000 nm taking into consideration the dissolution rate and the time required for the restoration of the vessel wall.

32. The stent according to claim 17, wherein the coating is created with a layer thickness in the range of 1 nm to 50 nm taking into consideration the dissolution rate and the time required for the restoration of the vessel wall.

33. The stent according to claim 17, wherein the layer thickness of the coating on the surface of the struts varies.
